(19) United States
(12) Patent Application Publication
(10) Pub. No.: US 2004/0175742 A1
(43) Pub. Date: Sep. 9, 2004

(54) Biosensor and method for detecting macromolecular biopolymers using at least one unit for immobilizing macromolecular biopolymers

(75) Inventors: Franz Hofmann, Munchen (DE); Richard Johannes Luyken, Munchen (DE); Petra Theresia Schindler-Bauer, Vaterstetten (DE)

Correspondence Address: DARBY & DARBY P.C., P. O. BOX 5257, NEW YORK, NY, 10150-5257, US

(73) Assignee: Infineon Technologies AG, Munich (DE)

(21) Appl. No.: 10/771,239

(22) Filed: Feb. 2, 2004

Related U.S. Application Data
(63) Continuation of application No. PCT/DE02/02706, filed on Jul. 23, 2002.

(30) Foreign Application Priority Data
Jul. 31, 2001 (DE) 101 37 342.2

(51) Int. Cl. C12Q 1/68

(52) U.S. Cl. 435/6

(57) ABSTRACT

A method for detecting macromolecular biopolymers using a macromolecular biopolymer immobilizing unit integrated in or mounted on a substrate. The macromolecular biopolymer immobilizing unit is provided with capture molecules that bind macromolecular biopolymers. A sample is brought into contact with the macromolecular biopolymer immobilizing unit, and the sample contains the macromolecular biopolymers to be detected and bound to the capture molecules. Any capture molecules to which no macromolecular biopolymers have bound are removed, and then generation of a chemiluminescence signal is induced using a label located on the capture molecules. The chemiluminescence signal is detected using a detection unit, which is an integrated circuit in the substrate, resulting in the macromolecular biopolymers being detected.

### CROSS-REFERENCE TO RELATED APPLICATION

This application is a continuation of International Patent Application Serial No. PCT/DE02/02706, filed Jul. 23, 2002, which published in German on Feb. 20, 2003 as WO 03/014695, and is incorporated herein by reference in its entirety.

### FIELD OF THE INVENTION

The invention relates to a biosensor and a method for detecting macromolecular biopolymers using at least one unit for immobilizing macromolecular biopolymers.

### BACKGROUND OF THE INVENTION

R. Hintsche et al., Microbiosensors Using Electrodes Made in Si-Technology, Frontiers in Biosensorics, Fundamental Aspects, edited by F. W. Scheller et al., Dirk Hauser Verlag, Basle, pp. 267-283, 1997, R. Hintsche et al., Microelectrode arrays and application to biosensing devices, Biosensors & Bioelectronics, Vol. 9, pp. 697-705, 1994, M. Paeschke et al., Voltammetric Multichannel Measurements Using Silicon Fabricated Microelectrode Arrays, Electroanalysis, Vol. 7, No. 1, pp. 1-8, 1996, and P. van Gerwen, Nanoscaled Interdigitated Electrode Arrays for Biochemical Sensors, IEEE, International Conference on Solid-State Sensors and Actuators, Chicago, pp. 907-910, 16-19 Jun. 1997, disclose methods for detecting DNA molecules in which biosensors which are based on electrode arrangements are used for the detection.

FIG. 4A and FIG. 4B show a sensor as described in the above-cited articles. The sensor 400 possesses two electrodes 401, 402 which are made of gold and which are embedded in an insulating layer 403 composed of insulating material. Electrode junctions 404, 405, to which the electric potential which is applied to the electrode 401, 402 can be supplied, are connected to the electrodes 401, 402. The electrodes 401, 402 are arranged as planar electrodes. DNA probe molecules 406 are immobilized on each electrode 401, 402 (see FIG. 4A). The immobilization is effected using what is termed gold-sulfur coupling. The analyte 407 to be investigated is applied to the electrodes 401, 402. In this connection, the analyte can, for example, be an electrolytic solution of different DNA molecules.

If the analyte 407 contains DNA strands 408 having a sequence which is complementary to the sequence of the DNA probe molecules 406, these DNA strands 408 then hybridize with the DNA probe molecules 406 (see FIG. 4B).

Hybridization of a DNA probe molecule 406 and of a DNA strand 408 only takes place when the sequences of the respective DNA probe molecule 406 and the corresponding DNA strand 408 are complementary to each other. If this is not the case, there is no hybridization. Consequently, a DNA probe molecule having a given sequence is in each case only able to bind, i.e. hybridize with, a particular DNA strand, namely the DNA strand having in each case the complementary sequence.

If a hybridization takes place, the capacity between the electrodes, in addition to other electrical parameters, changes, as can be seen from FIG. 4B. This change in capacity can be used as a measurable variable for detecting DNA molecules.

Furthermore, both of the R. Hintsche et al. articles and the M. Paeschke et al. article disclose a reduction/oxidation recycling method for detecting macromolecular biopolymers. In this method, a redox-active label is located on the proteins which are, for example, to be detected. After the proteins to be detected have been bound to capture molecules, this label then triggers a cycle of oxidation and reduction of suitable molecules, which cycle leads to an electric cycle current which is used for detecting the proteins.

However, optical methods, which are based on using fluorescent dyes, are chiefly used for detecting macromolecular biopolymers such as DNA molecules. Thus, N. L. Thompson, B. C. Lagerholm, Total Internal Reflection Fluorescence: Applications in Cellular Biophysics, Current Opinion in Biotechnology, Vol. 8, pp. 58-64, 1997, for example, discloses a procedure for examining the electrolyte for the existence of a DNA strand which possesses a given sequence. In this procedure, the DNA strands having the desired sequence are labeled with a fluorescent dye and their existence is determined with the aid of the fluorescence properties of the labeled molecules. To do this, the electrolyte is irradiated with light, which is in the visible or ultraviolet wave length range, for example, and the light which is emitted by the analyte, in particular by the labeled DNA strand which is to be identified, is detected. The fluorescence behavior, i.e., in particular, the emitted light rays which are detected, is used to determine whether the DNA strand which is to be identified and which possesses the appropriate given sequence is or is not present in the analyte.

This procedure is very elaborate since it is necessary to have a very precise knowledge of the fluorescence behavior of the corresponding label molecule on the DNA strand and, in addition to this, it is necessary to carry out a reaction for labeling the DNA strands before beginning the method. Furthermore, it is necessary to adjust the means for detecting the emitted light rays very precisely so as to ensure that these light rays can at all be detected.

In general, the detection and identification methods which operate on the basis of fluorescent dyes are disadvantageous insofar as the fluorescent radiation is detected using an external spectrometer. These spectrometers are expensive and elaborate to operate.

### SUMMARY OF THE INVENTION

The invention is based on the problem of providing an alternative method, and a device, for detecting macromolecular biopolymers.

The problem is solved by the method and the biosensor possessing the features described in the independent patent claims.

This method for detecting macromolecular biopolymers uses at least one unit for immobilizing macromolecular biopolymers, which unit is integrated in a substrate or applied on a substrate.

In the method, the at least one unit for immobilizing macromolecular biopolymers is provided with capture molecules, with the capture molecules being able to bind macromolecular biopolymers. A sample is then brought into contact with the at least one unit for immobilizing macromolecular biopolymers. In this connection, the sample can contain the macromolecular biopolymers which are to be detected. Macromolecular biopolymers which are present in the sample are bound to the capture molecules. In the method, a label is then used to excite the generation of a chemiluminescence signal. This chemiluminescence signal is detected by a detection unit which is designed as a circuit which is integrated in the substrate. This thereby detects the macromolecular biopolymers.

Expressed in simple terms, the present method is based on the realization that an “on-chip” detection using chemiluminescence labels or chemiluminescence radiation offers several advantages. In the first place, it offers the advantage that it is possible to use the entire chemiluminescence signal for detecting the macromolecular biopolymers and that, in this connection, in contrast to fluorescence-based detection methods, the emitted radiation does not have to be separated from the excitation radiation. In the second place, as a result of the signal detection and, where appropriate, the further subsequent processing of the signal, it is possible to dispense with external detection units such as spectrometers. This makes it possible to markedly simplify, and reduce the size of, the apparatus set-up. In the third place, the “on-chip” signal detection makes it possible to obtain site-resolved measurement.

The biosensor for detecting macromolecular biopolymers, which is disclosed here, possesses at least one unit for immobilizing macromolecular biopolymers, which unit is integrated in a substrate or applied on the substrate, and also a detection unit. In the biosensor, the at least one unit for immobilizing macromolecular biopolymers is provided with capture molecules. In this connection, the capture molecules can bind macromolecular biopolymers and possess a label which can generate a chemiluminescence signal. Furthermore, in the biosensor, the detection unit is designed as a circuit which is integrated in the substrate. In addition, the detection unit is designed such that it detects macromolecular biopolymers, which have become bound to the capture molecules, using the signal which is emitted by the label.

In one embodiment of the biosensor, the detection unit for detecting the optical chemiluminescence signal possesses at least one photodiode, CCD camera or CMOS camera.

In another embodiment, the biosensor possesses several units for immobilizing macromolecular biopolymers in a regular arrangement (an array). In the sensor, the at least one immobilizing unit, or the regular arrangement of the units, is placed on the photodiode, the CMOS camera or the CCD camera, i.e. above these detection units. If use is made of a regular arrangement of the detection units, e.g., photodiode panels or arrays, one or more immobilizing units can be placed on each detection unit, e.g., each photodiode.

In the method which is described here, the label is used to generate a chemiluminescence signal. The labels employed can be any labels which, as the result of a chemical reaction, directly or indirectly bring about the emission of an optical signal.

An example of a label which is by itself (i.e., directly in the present sense) able to bring about the generation of chemiluminescence radiation if horseradish peroxidase, which catalyzes the oxidation of cyclic diacyl hydrazides, such as luminol, in the presence of hydrogen peroxide (H₂O₂). This chemical reaction forms a reaction product in an excited state, with this product then passing into the ground state as a result of light emission. This light emission can be amplified by additional chemical coreactants, for example by 4-iodophenol in the case of the peroxidase/luminol system. Another example is Photinus pyralis luciferase which, in the presence of ATP and oxygen, catalyzes the conversion, with light emission, of luciferin into oxidized luciferin. Another example is alkaline phosphatase, which uses suitable 1,2-dioxetanes as substrates; see Roche Molecular Biochemicals, 1999 Biochemicals Catalog, p. 99. Such a label can, if desired, be linked directly to one of the two binding partners (the capture molecule or the biopolymer to be detected).

On the other hand, it is also possible, however, to make use, as a label, of a chemical compound which itself is unable to initiate any chemiluminescence reaction but which, however, possesses specific binding affinity for a binding partner which, for its part, is, for example, coupled to an enzyme such as horseradish peroxidase. Biotin is an example of such a label. This molecule possesses a high binding affinity for the protein streptavidin. If, for example, streptavidin is coupled to the abovementioned horseradish peroxidase, this reagent is then able, on the one hand, to bind to biotin, which has been incorporated as a label, for example into a biopolymer to be detected, and, on the other hand, to initiate a chemiluminescence reaction. It is evident from this that compounds such as biotin, avidin or digoxigenin can also be used, in the present case, as a label/label component for the capture molecules or the biopolymers to be detected.

The use of these indirectly operating labels can be advantageous since, metaphorically speaking, they stand at the beginning of a signal amplification cascade and are therefore able to increase the detection sensitivity of the method.

Within the meaning of the invention, detection is understood as being both the qualitative and quantitative detection of macromolecular biopolymers in an analyte (which is to be investigated). This means that the term “detection” also includes establishing the absence of macromolecular biopolymers from the analyte.

Within the meaning of the invention, “immobilizing unit” is understood as meaning an arrangement which possesses a surface on which the capture molecules can be immobilized, i.e., to which the capture molecules can bind by means of physical or chemical interaction. These interactions include hydrophobic or ionic (electrostatic) interactions and covalent bonds. Examples of suitable surface materials, which can be used for the at least one immobilizing unit, are metals, such as gold or silver, plastics, such as polyethylene or polypropylene, or inorganic substances, such as silicon dioxide, e.g., in the form of glass.

An example of a physical interaction which brings about immobilization of the capture molecules is adsorption to the surface. This type of immobilization can take place, for example, when the means for the immobilization is a plastic material which is used for preparing microtiter plates (e.g., polypropylene). However, preference is given to the capture molecules being linked covalently to the immobilizing unit because this makes it possible to regulate the orientation of the capture molecules. The covalent linkage can be effected using any suitable linking chemistry (“linker chemistry”). In one embodiment of the method, the at least one immobilizing unit is applied to an electrode or a photodiode.

The detection unit of the biosensor which is used here is designed as a circuit which is integrated in the substrate. This means that the immobilizing unit is either arranged on the same substrate or is integrated in this (common) substrate. This includes the possibility that the at least one immobilizing unit is arranged, for example, in a unit for receiving the substrate. An example of a suitable substrate is, for example, a semiconductor chip, in particular a CMOS chip or a silicon wafer. A receiving unit can, for example, be a housing or a mounting which, for example, receives a substrate such as a semiconductor chip. In this connection, the detection unit can be arranged in any arbitrary spatial orientation, with respect to the immobilizing unit, which makes it possible to detect a chemiluminescence signal which is generated by the label on/above or in the immediate vicinity of the immobilizing unit. In general, any material, in particular any semiconductor material, in which the detection unit can be embedded as a circuit is suitable for use as a substrate.

In one embodiment of the method, the chemiluminescence signal is generated by a label which is located on the macromolecular biopolymers which are to be detected.

In an alternative embodiment of the method, the chemiluminescence signal is generated by a label which is located on the capture molecules. This embodiment offers the advantage, in particular, that the macromolecular biopolymers to be detected do not previously have to be labeled to enable them to be detected. This avoids the danger of a part of the sample, or the entire sample, possibly being lost during the labeling reaction or of the labeling reaction not proceeding to completion, thereby falsifying the result.

In this embodiment, capture molecules to which no macromolecular biopolymers to be detected have bound are removed before the label is excited for generating the chemiluminescence signal.

The macromolecular biopolymers which the present methods can be used to detect are, in particular, nucleic acids, oligonucleotides, proteins or complexes composed of nucleic acids and proteins.

In a preferred embodiment of the method, the at least one immobilizing unit is placed on the detection unit, e.g., directly above the detection unit.

In a further development, several immobilizing units are placed in a regular arrangement on several detection units.

Preference is given to a method in which a photodiode, a CCD camera or a CMOS camera is used as the detection unit or the several detection units. In a preferred embodiment, a photodiode is used as the detection unit.

In one embodiment of the method, an electric signal, which is the consequence of the first step of the detection unit, which step converts the chemiluminescence signal into an electric signal, is used for detecting the biopolymers. Speaking generally, this electric signal is consequently induced indirectly by the chemiluminescence signal.

This electric signal produced by (in) the detection unit is preferably an electric current such as a photocurrent or a voltage such as a photovoltage in the case of the photodiode. The detection of the optical chemiluminescence signal, for example by integrating the electric signal, is preferably carried out over several minutes.

An advantage of the method, particularly in the case of multiple or parallel determinations, is that each individual detection unit for detecting the electric signal can be actuated individually. This thereby avoids any falsification of the measurement result, for example as the result of incident stray irradiation of adjacent sensor panels.

It may be pointed out here that, because of the omission of a detection device, such as a confocal microscope or an X-ray film, which is arranged outside of the reaction region, the configuration of the biosensor which is disclosed here does not only offer a simplified construction. Rather, the construction which is described here renders continuous measurement possible in the case of each immobilizing unit. This is particularly important and advantageous when, for example, processes which relate to the reaction dynamics or kinetics are to be investigated.

In another advantageous embodiment of the method, a nanoparticle is used as a unit for immobilizing macromolecular biopolymers.

Within the meaning of the invention, a nanoparticle is understood as being a particle which can be obtained using what are termed nanostructuring methods. Nanostructuring methods, which can be used for producing such nanoparticles on suitable substrates, are, for example, the use, which is described in J. P. Spatz et al., Mineralization of Gold Nanoparticles in a Block Gold Copolymer Microemulsion. Chem. Eur. J., Vol. 2, pp. 1552-1555, 1996, and J. P. Spatz et al., Ordered Deposition of Inorganic Cluster from Micellar Block Copolymer Films, Langmuir, Vol. 16, pp. 407-415, 2000, of block copolymer microemulsions, or the use, which is described in F. Burmeister et al., Mit Kapillarkräften zu Nanostrukturen [With capillary forces to nanostructures], Physikalische Blätter [physics Pages], Vol. 36, pp. 49-51, 2000, of colloidal particles as structuring masks. The method described in F. Burmeister et al. is in principle analogous to a lithographic method which is customarily used in the field of substrate structuring. It may therefore be emphasized, at this point, that, within the meaning of the invention, a nanoparticle is consequently not restricted to those particles which are obtained using one of the methods which are mentioned here by way of example. On the contrary, such a nanoparticle is any particle whose diameter is in the nanometer range, i.e., generally in the range from 2 to 50 nm, preferably in the range from 5 to 20 nm, particularly preferably in the range from 5 to 10 nm.

An “immobilizing unit which is a nanoparticle”, and which is also termed a nanoparticle-shaped unit in that which follows, is consequently an above-described nanoparticle which possesses a surface on which the capture molecules can be immobilized, i.e., the surface is constituted such that the capture molecules are able to bind to it by means of physical or chemical interactions. These interactions include hydrophobic or ionic (electrostatic) interactions and covalent bonds. Examples of suitable surface materials which can be used for the at least one nanoparticle-shaped immobilizing unit are metals, such as gold or silver, semiconducting materials, such as silicon, plastics, such as polyethylene or polypropylene, or silicon dioxide, for example in the form of glass. In this connection, nanoparticle-shaped units composed of plastics and silicon dioxide can be obtained by using the colloid mask method described in F. Burmeister et al.. Nanoparticle-shaped units composed of semiconducting materials such as silicon can also be formed, for example, using the Stranski-Krastanov method. It is furthermore possible to obtain nanoparticle-shaped units composed of silicon dioxide by oxidizing such nanoparticles composed of silicon.

On account of the above-described preparation methods, nanoparticle-shaped immobilizing units which are applied on suitable substrate surfaces (retaining regions), for example of photodiodes or CCDs, adopt a regular arrangement, with distances from each other in the region of some tens of nanometers, for example from approx. 10 to 30 nm on these surfaces. The nature of the arrangement, and the distance of the nanoparticles from each other, depends, like the size of the nanoparticles, on the particular method used for forming the nanoparticles.

An advantage of using nanoparticle-shaped immobilizing units is that it is possible to immobilize a precisely defined number of capture molecules on these nanoparticles. This is particularly advantageous when using the present method for quantitatively detecting macromolecular biopolymers. Another advantage of using nanoparticles as immobilizing units arises from the fact that the distance of the nanoparticles from each other, i.e., the spatial separation of the capture molecules, provides a superior spatial accessibility of the capture molecules for the macromolecular biopolymers which are binding to them and consequently increases the probability of an interaction. Moreover, the configuration as nano- particles enlarges the effective surface.

In this present case, macromolecular biopolymers are understood as meaning, for example, (relatively long-chain) nucleic acids, such as DNA molecules, RNA molecules, PNA molecules or cDNA molecules, or relatively short oligonucleotides containing, for example, from 10 to 50 base pairs (bp), in particular from 10 to 30 bp. While the nucleic acids may be double-stranded, they also at least possess single-stranded regions or are present, for their detection, as single strands, for example as a result of previous thermal denaturation (strand separation). In this connection, the sequence of the nucleic acids to be detected can be at least partially, or completely, predetermined, i.e., known. Other macromolecular biopolymers are proteins or peptides. While these latter can be composed of the 20 amino acids which are customarily found in proteins, they can also contain amino acids which do not naturally occur or can, for example, be modified with sugar residues (oligosaccharides) or contain post-translational modifications. It is furthermore also possible to detect complexes which are composed of several different macromolecular biopolymers, for example complexes composed of nucleic acids and proteins.

If the macromolecular biopolymers to be detected are proteins or peptides, preference is then given to using ligands which are able to specifically bind the proteins or peptides to be detected as the capture molecules. The capture molecules/ligands are preferably linked to the immobilizing unit by means of covalent bonds.

Suitable ligands for proteins and peptides are low molecular weight enzyme agonists or enzyme antagonists, pharmaceuticals, sugars or antibodies, or other suitable molecules which have the ability to specifically bind proteins or peptides.

When DNA molecules (nucleic acids or oligonucleotides) having a predetermined nucleotide sequence are detected using the method which is described here, they are preferably detected in single-stranded form, i.e., they are converted, where appropriate prior to the detection, into single strands by denaturation, as explained above. In this case, preference is then given to using, as capture molecules, DNA probe molecules which possess a sequence which is complementary to the single-stranded region. The DNA probe molecules can in turn exhibit oligonucleotides or longer nucleotide sequences as long as the latter do not form any of the intermolecular structures which prevent hybridization of the probe molecule with the nucleic acid to be detected. However, it is also possible to employ DNA-binding proteins or agents as the capture molecule.

It is to be noted that it is naturally not the case that it is only possible to use the present method to detect one type of biopolymer in a single measurement series. On the contrary, it is possible to detect several macromolecular biopolymers simultaneously or else consecutively. To do this, several types of capture molecule, each of which has a (specific) binding affinity for a particular biopolymer to be detected, can be bound on the immobilizing unit and/or several immobilizing units can be employed, with only one type of capture molecule being bound to each of these units. In these multiple determinations, preference is given to using, for each macromolecular biopolymer which is to be detected, a label which can be differentiated from the other labels by the wavelength of the chemiluminescence signal which is emitted.

In a first procedural step, the at least one immobilizing unit is provided with the capture molecules, with these molecules, in one embodiment of the method, possessing a label which can be used to produce the detectable signal.

A sample to be investigated, preferably a liquid medium such as an electrolyte, is then brought into contact with the immobilizing unit. This is effected such that the macromolecular biopolymers are able to bind to the capture molecules. If several macromolecular biopolymers to be detected are present in the medium, the conditions are selected such that these biopolymers are in each case able to bind to their corresponding capture molecule at the same time or consecutively.

After a sufficient period of time for the macromolecular biopolymers to be able to bind to the corresponding capture molecule or the corresponding capture molecules has been allowed to pass, unbound capture molecules are removed from the immobilizing unit, or units, on which they are located. In the embodiment of the method in which the capture molecules are provided with a label, this step is obligatory. However, unbound capture molecules can also be removed when the biopolymers to be detected are carrying the label.

If proteins or peptides are to be detected as the macromolecular biopolymers, the unbound ligands, which are preferably immobilized by way of a covalent bond and used as capture molecules, are removed from the at least one immobilizing unit by bringing a material into contact with the at least one immobilizing unit. In this connection, the material is able to hydrolyze the chemical bond between the ligand and the immobilizing unit.

When the capture molecules are low molecular weight ligands, they can, if unbound, also be removed enzymically.

In order to be able to do this, the ligands are bonded covalently to the immobilizing unit by way of an enzymically cleavable bond, for example by way of an ester bond.

In this case, it is possible, for example, to use a carboxylic ester hydrolase (esterase) in order to remove unbound ligand molecules. This enzyme hydrolyzes the ester bond between the immobilizing unit and the particular ligand molecule which has not been bound by a peptide or protein. On the other hand, the ester bonds between the immobilizing unit and those molecules which have entered into a binding interaction with peptides or proteins remain intact because of the decrease in steric accessibility due to space filling by the bound peptide or protein.

When the capture molecules are DNA strands, the unbound capture molecules are removed enzymically, for example using an enzyme which possesses nuclease activity. The enzyme possessing nuclease activity which is preferably used is an enzyme which selectively degrades single-stranded DNA. The selectivity of the degrading enzyme for single-stranded DNA has to be taken into account in this connection. If the enzyme which is chosen for degrading the unhybridized DNA single strands does not possess this selectivity, the DNA to be detected, which is present together with the capture molecule in the form of a double-stranded hybrid, may also possibly, and undesirably, be degraded.

In particular, it is possible to use DNA nucleases, for example a Mung bean nuclease, P1 nuclease or S1 nuclease, for removing the unbound DNA capture molecules, which are also termed probe molecules below, from the electrode in question. It is likewise possible to use DNA polymerases which are able to degrade single-stranded DNA on account of their 5′→3′ exonuclease activity or their 3′→5′ exonuclease activity.

After the unbound capture molecules have been removed, the macromolecular biopolymers are detected by the detection unit using the label.

The biosensor which is used for this purpose in this present case is configured such that the measurement takes place directly, in a site-resolved manner, on the sensor by, for example, the immobilizing unit being directly applied to a photocell which is used for the measurement. Furthermore, the sensor can possess switching elements for individually actuating each detection unit and a corresponding evaluation and signal-processing unit. In this connection, this evaluation unit can, in addition to the integrator for the electric signal, exhibit, for example, an analog/digital transformer and/or a preamplifier for detecting the electric signal. These units are preferably all integrated in the sensor.

This has the advantage of simplifying the measurement set-up. Such a measurement set-up can be implemented, for example, using a CMOS camera or a CCD.

In the method, a measurement of the signal can be carried out as a reference measurement before or after the at least one unit for immobilizing macromolecular biopolymers has been provided with the capture molecules. The measurement which is used for the detection is then carried out. In both cases, the values which are determined from the two measurements of the resulting (electric) signal are compared with each other. If the signal intensities of the measured values differ from each other such that the difference in the values determined is greater than a specified threshold value, it is then assumed that macromolecular biopolymers have bound to capture molecules, thereby causing the change in the intensity of the signal which is received at the detector. Then, if the value which is determined is smaller than the threshold value, it is assumed that no biopolymers have bound to the capture molecules, i.e., that these biopolymers were not present in an investigated sample, either.

### BRIEF DESCRIPTION OF THE DRAWINGS

Examples of implementing the invention are depicted in the figures and explained in more detail below.

FIGS. 1A to 1C show a biosensor at different procedural states which are used to explain the method in accordance with one example of implementing the invention;

FIGS. 2A to 2C show a biosensor at different procedural states which are used to explain the method in accordance with another example of implementing the invention;

FIGS. 3A to 3F show a biosensor which can be used to carry out another embodiment of the method which is described here;

FIGS. 4A and 4B show a sketch of two planar electrodes which can be used to detect the existence (FIG. 2A) or nonexistence (FIG. 2B) of DNA strands to be detected in an electrolyte.

### DETAILED DESCRIPTION OF THE PREFERRED MODE OF THE INVENTION

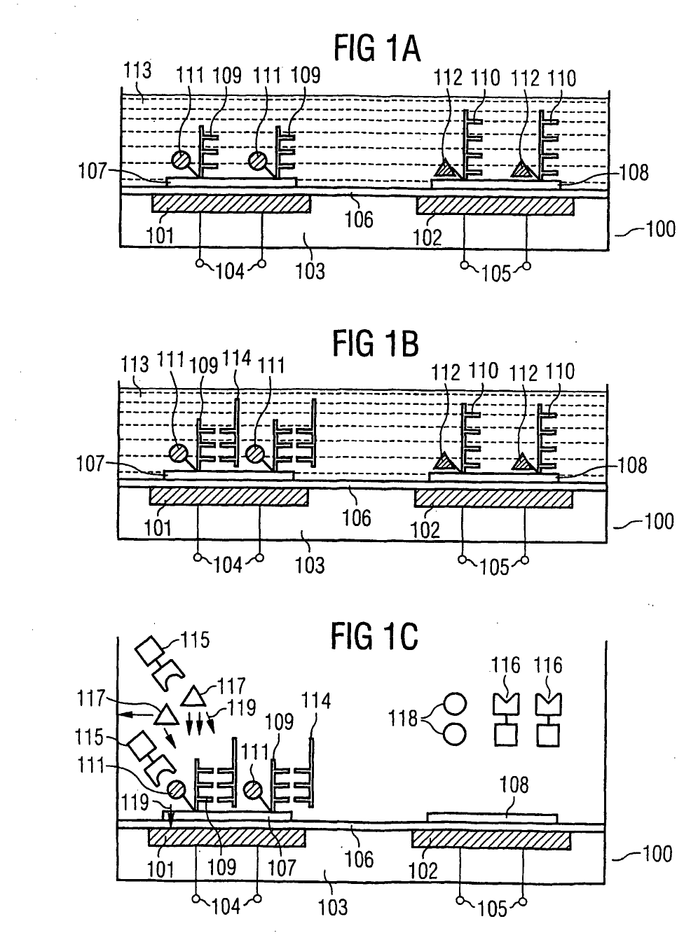

FIGS. 1A through 1C shows a detail from a biosensor 100 which can be used to carry out a first example of implementing the method which is described here.

FIG. 1A shows the biosensor 100 with a first photodiode 101 and a second photodiode 102 which are arranged in a layer 103 composed of semiconducting material.

The first photodiode 101 and the second photodiode 102 are connected by way of first electric junctions 104 and second electric junctions 105, respectively, to an evaluation unit (not depicted). This evaluation unit can be integrated in the sensor 100. In addition, the two photodiodes 101, 102 are provided with an oxide layer 106 and a first unit 107 for immobilizing macromolecular biopolymers and, respectively, a second unit 108 for immobilizing macromolecular biopolymers. The immobilizing units 107 and 108 are made of gold.

Alternatively, the immobilizing units 107, 108 can also be made of silicon oxide and coated with a material which is suitable for immobilizing capture molecules.

For example, it is possible to use known alkoxysilane derivatives, such as 3-glycidoxypropylmethyloxysilane, 3-acetoxypropyltrimethoxysilane, 3-aminopropyltriethoxysilane, 4-(hydroxybutyramido)propyltriethoxysilane, 3-N, N-bis(2-hydroxyethyl)aminopropyltriethoxysilane, or other related materials which are able to enter into a bond, for example a covalent bond, with the surface of the silicon oxide using one of their ends and to offer, to the probe molecule to be immobilized, for reaction, a chemically reactive group, such as an epoxy radical, acetoxy radical, amine radical or hydroxy radical, using their other end. Alternatively, it is possible, for example, to use poly-L-lysine.

If a capture molecule which is to be immobilized reacts with such an activated group, it is then bonded on the immobilizing unit by way of the chosen material as a type of covalent linker on the surface of the coating.

DNA probe molecules 109, 110 are applied, as capture molecules, on the immobilizing units 107 and 108.

In this connection, first DNA probe molecules 109 having a sequence which is complementary to a given first DNA sequence are applied to the first photodiode 101 by means of the unit 107. The DNA probe molecules 109 are in each case provided with a first chemiluminescence signal-generating label 111.

Biotin, which can be incorporated both at the 5′ terminus and at the 3′ terminus of a DNA capture molecule 109, can be used as the label; see IBA Product Guide 2001, page 81.

Second DNA probe molecules 110 having a sequence which is complementary to a given second DNA sequence are applied to the second photodiode 102. The DNA probe molecules 110 are in each case labeled with a second label 112. Digoxigenin can, for example, be used as the label 112 in this connection; see Roche Molecular Biochemicals, 1999 Biochemicals catalog, p. 89.

Sequences of DNA strands, which sequences are in each case complementary to the sequences of the probe molecules, can hybridize in the customary manner, i.e., by means of base pairing, by way of hydrogen bonds, between A and T or between C and G, respectively, at the purine bases adenine (A) and guanine (G) and pyrimidine bases thymine (T) or uracil (U) in the case of an above-described label, or cytosine (C). When other nucleic acid molecules are used, other bases are correspondingly used, for example uridine (U) in the case of an RNA molecule.

FIG. 1A furthermore shows an electrolyte 113 which is brought into contact with the photodiodes 101, 102 and the DNA probe molecules 109, 110.

FIG. 1B shows the biosensor 100 for the situation in which the electrolyte 113 contains DNA strands 114 which possess a given first nucleotide sequence which is complementary to the sequence of the first DNA probe molecules 109.

In this case, the DNA strands 114 which are complementary to the first DNA probe molecules 109 hybridize with the first DNA probe molecules 109 which are loaded on the first photodiode 101.

Since the sequences of DNA strands only hybridize with the complementary sequence which is in each case specific, the DNA strands which are complementary to the first DNA probe molecules do not hybridize with the second DNA probe molecules 110.

As can be seen from FIG. 1B, the result, after hybridization has taken place, is that hybridized molecules are located, i.e., double-stranded DNA molecules are immobilized, on the first photodiode 101. Only the second DNA probe molecules 110 are present, as molecules which are still single-stranded, on the second photodiode 102.

In a further step, hydrolysis of the single-stranded DNA probe molecules 110 on the second photodiode 102 is brought about by means of a (biochemical) method, for example by adding DNA nucleases to the electrolyte 113.

In this connection, the selectivity of the degrading enzyme for single-stranded DNA has to be considered. If the enzyme which is chosen for degrading the unhybridized DNA single strands does not possess this selectivity, the nucleic acid to be detected, which is present as double-stranded DNA, may possibly also (undesirably) be degraded, an event which would lead to falsification of the measurement result.

After the single-stranded DNA probe molecules, i.e., the second DNA probe molecules 110 on the second photodiode 102, have been removed, it is only the hybrids, composed of the DNA molecules 114 to be detected and the first DNA probe molecules 109 which are complementary to them, which are present (see FIG. 1C).

As examples, one of the following substances can be added in order to remove the unbound single-stranded DNA probe molecules 110 on the second photodiode 102, i.e., the second immobilizing unit: Mung bean nuclease, P1 nuclease, or S1 nuclease.

DNA polymerases which, on account of their 5′→3′ exonuclease activity or their 3′→5′ exonuclease activity, are able to degrade single-stranded DNA can also be used for this purpose.

After that, a chemical conjugate 115, 116 which is suitable for detecting the label 111 and 112, respectively, is added, together with the corresponding respective substrate 117, 118 and, where appropriate, signal amplifiers, in order, in this way, to generate a chemiluminescence signal (FIG. 1C, in which figure the electrolyte 113, or the reaction medium which is required for the detection reaction, is not shown for reasons of clarity). In the case of the biotin label 111, it is possible, for example, to use a conjugate 115 which is composed of streptavidin and horseradish peroxidase, with luminol being used as the substrate and 4-iodophenol being used as the signal amplifier. In the case of the digoxigenin label 112, it is possible, for example, to use what is termed an “anti-digoxigenin-alkaline phosphate conjugate”, with CSPD® being used as the chemiluminescence substrate (Roche Molecular Biochemicals, 1999 Biochemicals catalog, p. 89.) The detection reactions can be carried out in parallel or consecutively depending on the compatibility of the reaction conditions. When carrying out parallel determinations, the biosensor can also be designed in such a way that a spatial separation into separate reaction chambers is achieved using, for example, gates and walls.

It is only in the case of the label 111 that adding the detection conjugate excites the label which is located on the first DNA probe molecules 109 to generate the chemiluminescence signal since the unbound second DNA probe molecules 110, together with the label 112, have been removed from the second photodiode 102 by the nuclease treatment (see FIG. 1C). The emitted chemiluminescence radiation, which is symbolized by the arrow 119, is detected by the first photodiode 101. On the other hand, no chemiluminescence radiation is detected at the second photodiode 102.

In this way, the presence of the DNA molecules 114 is determined. The use of the bio sensor 100 which is described here permits site-resolved detection and provides a marked simplification of the overall measurement set-up since no external unit is required for detecting an optical signal such as fluorescence radiation or chemiluminescence radiation.

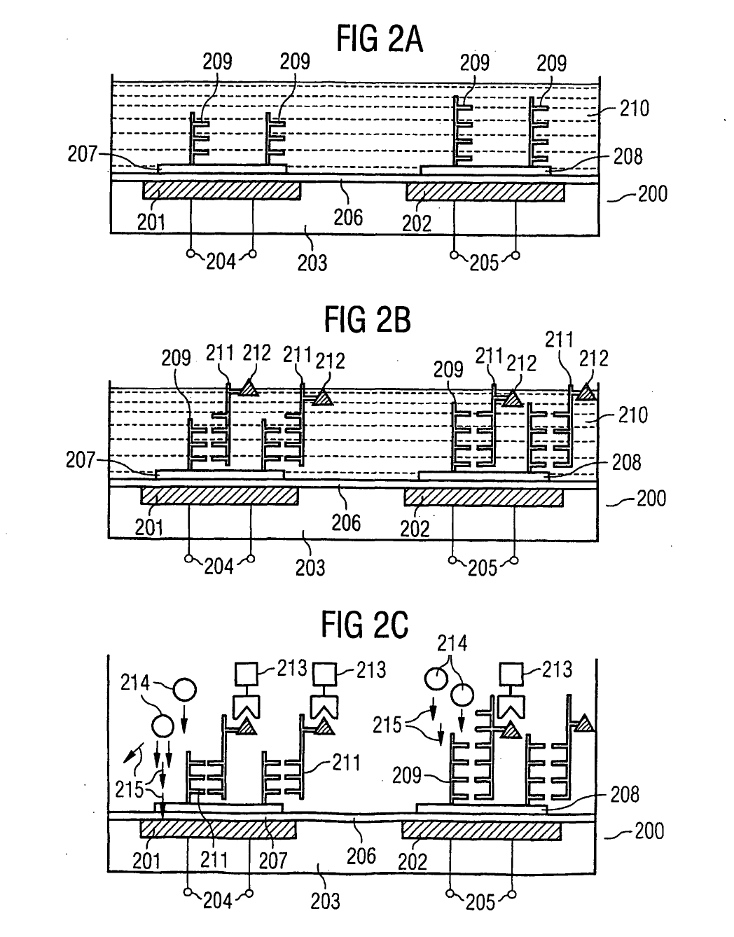

FIG. 2a shows the biosensor 200, whose construction is identical to that of the biosensor 100. That is, the sensor 200 possesses a first photodiodes 201 and a second photodiode which are arranged in a layer 203 composed of semiconducting material.

The first photodiode 201 and a second photodiode 202 are connected to an evaluation unit (not depicted) by way of first electric junctions 204 and second electric junctions 205, respectively. This evaluation unit may be integrated in the sensor 200. In addition, the two photodiodes 201, 202 are provided with an oxide layer 206 and a first unit 207 for immobilizing macromolecular biopolymers or, respectively, a second unit 208 for immobilizing macromolecular biopolymers. The immobilizing units 207 and 208 are made, for example, from gold or silicon. DNA probe molecules 209 are loaded on them, as capture molecules, in the manner described above.

In this connection, first DNA probe molecules 209, having a sequence which is complementary to a given (first) DNA sequence, are, in this example, loaded on to both the first photodiode 201 and the second photodiode using the units 207 and 208, respectively.

FIG. 2A furthermore shows an electrolyte 210, which is brought into contact with the photodiodes 201, 202 and the DNA probe molecules 209.

FIG. 2B shows the biosensor 200 when the electrolyte 210 contains DNA molecules 211 which possess a given first nucleotide sequence which is complementary to the sequence of the first DNA probe molecules 209. The DNA molecules 211 carry, as labels 212, the above-described digoxigenin label or biotin label, for example.

In this case, hybrids composed of the DNA probe molecules 209 and the DNA molecules 211 which are complementary to them, are formed on the photodiodes 201, 202 (FIG. 2B).

After a washing step, which is carried out where appropriate, a conjugate 213, which is suitable for detecting the label 212, together with a substrate 214 and, where appropriate, a signal amplifying compound, are added, as described above, in order to generate a chemiluminescence signal.

In connection with this, chemiluminescence radiation is emitted, as symbolized by the arrow 215, and this radiation is detected by the photodiodes 201, 202. This thereby determines the presence of the DNA molecules 211.

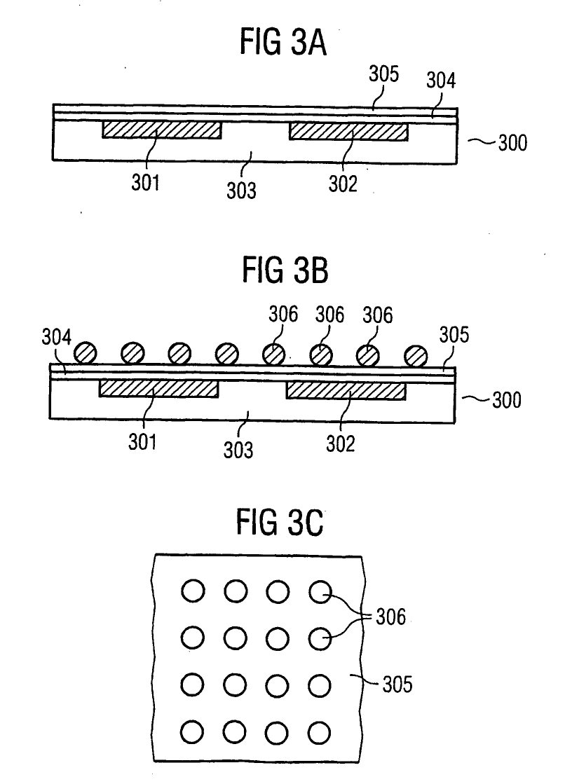

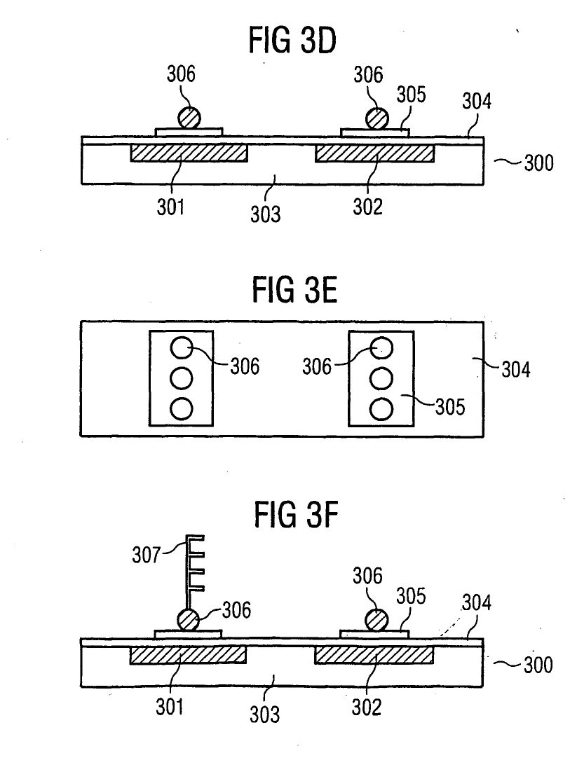

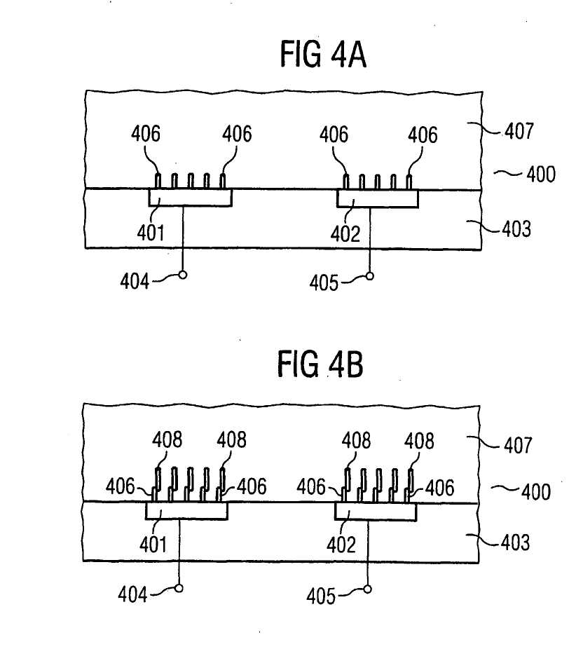

FIGS. 3A through 3F show a detail of a biosensor 300 which is designed with at least one immobilizing unit in the form of nanoparticles and which can be used to carry out another embodiment of the method which is described here.

The biosensor 300 possesses a first photodiode 301 and a second photodiode 302 which are arranged in a layer 303 composed of semiconducting material such as silicon. The biosensor 300 furthermore possesses an oxide layer 304 and a second layer 305 which is located thereon. The second layer 305 consists of a metal which is not suitable for immobilizing macromolecular biopolymers. This layer 305 can be formed, for example, from platinum.

The units for immobilizing macromolecular biopolymers, which units have the form of nanoparticles, are formed on the layer 305 using the following method.

A solution of 0.5% by weight of polystyrene (PS)-block-poly(2-vinylpyridine) (P2VP) block copolymer of the general formula PS(x)-b-P2VP(y) is treated, as described in the two J. P. Spatz et al. articles, with 0.5 equivalents of HAuCl₄.H₂O per pyridine unit in order to form monodisperse (dissolved as micelles) gold particles. In the formula, x and y indicate the number of basic units corresponding to the ratio between monomer and initiator.

After homogeneous micelles have been formed, a monolayer of gold nanoparticles is precipitated from this solution onto the layer 305 by reducing with hydrazine, as described in the two J. P. Spatz et al. articles. The organic constituents of the precipitated micelles, i.e., the block copolymer, are then removed from the layer 305 by means of plasma etching using an oxygen plasma (see J. P. Spatz et al., Ordered Deposition of Inorganic Cluster from Micellar Block Copolymer Films, Langmuir, Vol. 16, pp. 407-415, 2000). In this treatment with plasma, the gold particles 306, which serve as the units for immobilizing macromolecular biopolymers, remain undamaged and form a regular arrangement on the layer 305, as illustrated in the sectional view seen in FIG. 3B and the view from above seen in FIG. 3C (see J. P. Spatz et al., Mineralization of Gold Nanoparticles in a Block Gold Copolymer Microemulsion. Chem. Eur. J., Vol. 2, pp. 1552-1555, 1996). As a rule, the distances between the gold nanoparticles 306 amount to a few 10 nm, e.g., from approx. 20 to 30 nm. The size of the nanoparticles is preferably in the range from approx. 5 to 10 nm.

Aside from the abovementioned block polymers, it is naturally also possible to use other block polymers for forming the nanoparticles.

Alternatively, the immobilizing unit 306 can be produced in nanoparticle form on the biosensor as described in F. Burmeister et al. by initially forming a mask for the nanostructuring out of colloidal particles on the layer 305 and then depositing gold particles by means of vacuum deposition, for example.

After the gold nanoparticles 306 have been applied, the sensor 300 is structured such that the layer 305 composed of platinum and the immobilizing units 306 only remain on regions which are located on the photodiodes 301, 302, as shown in the sectional view seen in FIG. 3D and the view from above seen in FIG. 3E. This structuring can be effected by, for example, using any suitable standard chemical etching method.

The biosensor 300 which has been configured in this way can be used, for example, to carry out both the methods for detecting macromolecular biopolymers which are described in the implementation examples shown in FIG. 1. FIG. 3F shows a DNA capture molecule 307 which has been immobilized by means of gold-sulfur coupling on a gold nanoparticle 306. This capture molecule can, if desired, be provided with a label which is used to generate a chemiluminescence signal.

The use of the biosensor 300 provides the advantage that the immobilizing units 305 which are present in nanoparticle form enable a precisely defined number of capture molecules to be immobilized. The use of the biosensor 300 is therefore preferred when macromolecular biopolymers are being detected quantitatively.

What is claimed is:

1. A method for detecting macromolecular biopolymers using a macromolecular biopolymer immobilizing unit integrated in or mounted on a substrate, the method comprising the steps of:
providing the macromolecular biopolymer immobilizing unit with capture molecules that bind macromolecular biopolymers;
bringing a sample into contact with the macromolecular biopolymer immobilizing unit, the sample containing the macromolecular biopolymers to be detected and bound to the capture molecules;
removing any capture molecules to which no macromolecular biopolymers have bound;
inducing generation of a chemiluminescence signal using a label located on the capture molecules; and
detecting the chemiluminescence signal using a detection unit, which is an integrated circuit in the substrate, resulting in the macromolecular biopolymers being detected.

2. The method as claimed in claim 1, wherein the macromolecular biopolymers detected are at least one of nucleic acids, oligonucleotides, proteins, and complexes composed of nucleic acids and proteins.

3. The method as claimed in claim 1, wherein the immobilizing unit is an array of nanoparticles.

4. The method as claimed in claim 1, wherein the immobilizing unit is provided on the detection unit.

5. The method as claimed in claim 4, further comprising the step of providing a plurality of immobilizing units mounted in a regular arrangement on several detection units.

6. The method as claimed in claim 4, wherein the detection unit is one of a photodiode, CCD camera or CMOS camera.

7. The method as claimed in claim 1, further comprising the step of providing a plurality of chemiluminescence signal detection units, wherein each detection unit is actuated individually.

8. A biosensor for detecting macromolecular biopolymers, comprising:
a substrate;
a macromolecular biopolymer immobilizing unit integrated in or mounted on the substrate and provided with capture molecules that bind biopolymers and exhibit a label which generates a chemiluminescence signal;
a detection unit, which is integrated in the substrate, that uses the signal generated by the label to detect macromolecular biopolymers which have bound to the capture molecules,
wherein capture molecules to which no macromolecular biopolymers to be detected have bound are removed before the generation of the chemiluminescence signal.

9. The biosensor as claimed in claim 8, wherein the detection unit includes at least one of a photodiode, CCD camera or CMOS camera.

10. The biosensor as claimed in claim 8, further comprising several macromolecular biopolymer immobilizing units in a regular arrangement.

11. The biosensor as claimed in claim 9, wherein the immobilizing unit is mounted on the detection unit.
